(19) United States
(12) Patent Application Publication
(10) Pub. No.: US 2025/0387201 A1
(43) Pub. Date: Dec. 25, 2025

(54) SYNTHETIC SUPRACRESTAL DENTAL IMPLANT FIBERS

(71) Applicants: New York University, NEW YORK, NY (US); Case Western Reserve University, Cleveland, OH (US)

(72) Inventors: Leena PALOMO, New York, NY (US); Steven J. EPPELL, Beachwood, OH (US)

(21) Appl. No.: 18/878,811

(22) PCT Filed: Jun. 30, 2023
(86) PCT No.: PCT/US2023/026726

§ 371 Date: Dec. 24, 2024

Related U.S. Application Data
(60) Provisional application No. 63/367,540, filed on Jul. 1, 2022.

(51) Int. Cl. A61C 8/02; A61C 8/00; A61K 6/69; A61K 6/84

(52) U.S. Cl. CPC A61C 8/0006; A61C 8/0012; A61C 8/0022; A61C 8/005; A61C 8/0077; A61K 6/69; A61K 6/84

(57) ABSTRACT

A dental implant for facilitating interdental papilla growth includes an implanted portion configured to be inserted into a jawbone; an abutment configured to be attached to an exposed end of the implanted portion; and supracrestal fibers extending laterally from an exterior surface of the abutment to form a scaffold between the dental implant and an adjacent tooth for facilitating interdental papilla growth in a supracrestal region of the dental implant.

### PRIORITY CLAIM

The present disclosure claims priority to U.S. Provisional Patent Application Ser. No. 63/367,540 filed Jul. 1, 2022; the disclosure of which is incorporated herewith by reference.

### BACKGROUND

Interdental papilla is the gum tissue found in the space between the teeth. It helps to protect the root of a tooth and keeps food from getting stuck between teeth, which may lead to decay. When a patient requires dental implants, however, the interdental papilla surrounding the tooth may be compromised. In some cases, when the gum tissue does not completely fill the space between adjacent teeth, “black triangles,” also known as open gingival embrasures, may be visible between the teeth. A lack of interdental papilla may be both aesthetically displeasing as well as detrimental to the patient's dental health. As dental implants have become more common, restoring and/or reconstructing the interdental papilla has become necessary, but remains difficult. Several approaches for restoring papilla have been attempted.

For example, in some cases the biology known about how the supracrestal environment creates the papilla has been studied. There would be cells that were involved, non-collagenous proteins involved, signaling factors, cell surface receptors, etc. A tissue engineer may, for example, in an attempt to recapitulate embryological processes, put in cells and complicated drug-delivery systems to get the cells to respond properly to build up the scaffold and make the papilla. Most people look at the entire problem from a cell structural and even bone morphogenic, soft tissue morphogenic, angiogenic type of cell remodeling point of view. Others try to introduce bone morphogenetic proteins and platelet-derived growth factor (PDGF). These approaches, however, are complicated, traumatic, time consuming and produce variable results.

### SUMMARY OF THE INVENTION

The present disclosure relates to a dental implant for facilitating interdental papilla growth, comprising an implanted portion configured to be inserted into a jawbone, an abutment configured to be adjacent to the exposed end of the implanted portion, and supracrestal fibers extending laterally from an exterior surface of the abutment to form a scaffold between the dental implant and an adjacent tooth for facilitating interdental papilla growth in a supracrestal region of the dental implant. In an exemplary embodiment the supracrestal fibers may form a bridge between the abutment of the dental implant and the gingiva (e.g., soft tissue) extending between the dental implant and the adjacent tooth.

In an embodiment, the supracrestal fibers are covalently attached to the abutment to extend substantially perpendicularly from the exterior surface of the abutment.

In an embodiment, the implanted portion is configured as a screw threadable into the jawbone.

In an embodiment, the dental implant further includes a tooth-shaped crown configured to be mounted over the abutment and adhered thereto.

In an embodiment, the abutment is formed of titanium.

In an embodiment, the abutment includes a smooth, machined surface.

In an embodiment, the implanted portion and the abutment are integrally formed.

In an embodiment, the supracrestal fibers are mineralized using fetuin.

In an embodiment, the supracrestal fibers are incorporated with one of growth factors, analgesics and antibiotics.

In addition, the present disclosure relates to a method for dental implantation. The method includes inserting an implanted portion into a jawbone, an abutment attached to an exposed end of the implanted portion so that the abutment extends exterior to the jawbone and supracrestal fibers extend laterally from the abutment to form a scaffold between the abutment and an adjacent tooth to facilitate growth of interdental papilla in a supracrestal region.

In an embodiment, the supracrestal fibers are covalently attached to the abutment to extend substantially perpendicularly from an exterior surface of the abutment.

In an embodiment, inserting the implanted portion includes threading a screw of the implanted portion into the jawbone.

In an embodiment, the method further includes mounting a tooth-shaped crown over the abutment and adhering the tooth-shaped crown thereto.

In an embodiment, the abutment is formed of titanium.

In an embodiment, the abutment includes a smooth, machined surface.

In an embodiment, the abutment is attached to the implanted portion after implantation of the implanted portion into the jawbone.

In an embodiment, the implanted portion and the abutment are integrally formed.

In an embodiment, the supracrestal fibers are mineralized using fetuin.

In an embodiment, the supracrestal fibers are incorporated with one of growth factors, analgesics and antibiotics.

### BRIEF DESCRIPTION

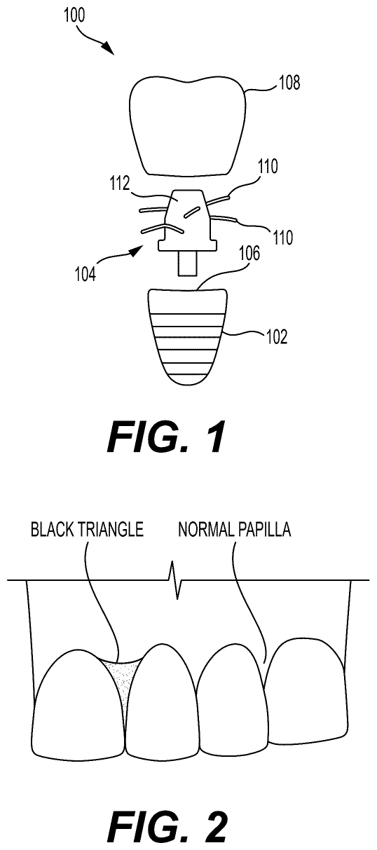

FIG. 1 shows an exploded view representative of a dental implant according to an exemplary embodiment of the present disclosure.

FIG. 2 shows a drawing of “black triangles” between adjacent teeth, also known as, open gingival embrasures.

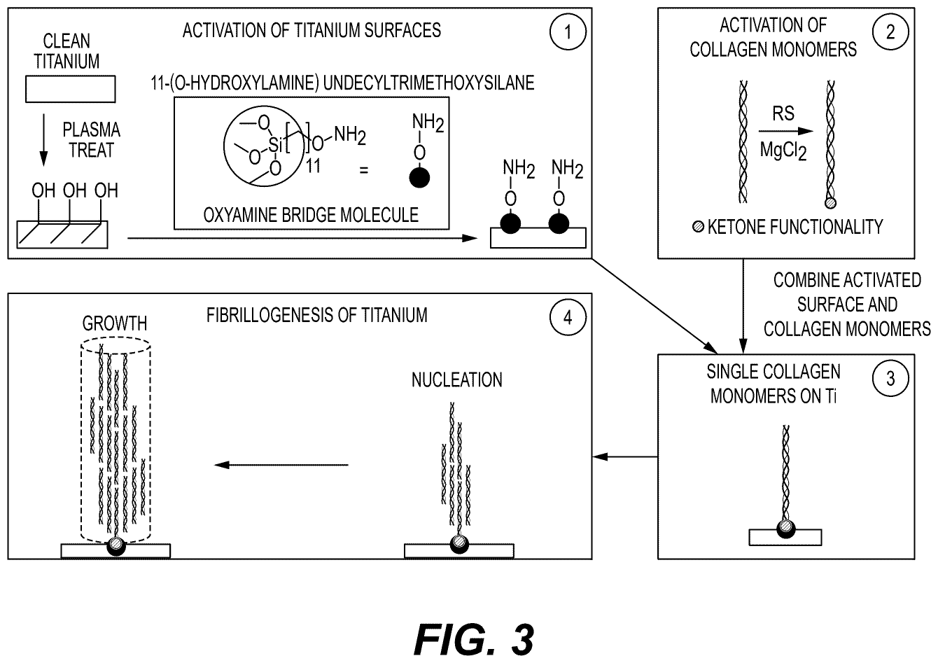

FIG. 3 shows a schematic diagram of a method for creating collagen fibers on a titanium surface.

### DETAILED DESCRIPTION

The present disclosure may be further understood with reference to the following description and the appended drawings, wherein like elements are referred to with the same reference numerals. The present disclosure relates to a system and method for inducing formation of papilla in the supracrestal regions around dental implants so as to relieve the compromised aesthetics and function which exists around current dental implants. For example, in some cases, a lack of interdental papilla will cause “black triangles” to be visible between adjacent teeth. This may be particularly problematic when two or more dental implants are placed adjacent to one another.

Exemplary embodiments describe forming one or more supracrestal fibers on a titanium abutment of a dental implant to form a scaffold between the dental implant and an adjacent tooth (e.g., another dental implant), which facilitates the regrowth of papilla there between. The supracrestal fibers are formed on an exterior surface of the abutment, substantially perpendicularly thereto, so that the scaffold is formed in a supracrestal region of the dental implant. This allows the supracrestal fibers to generate a healing response resulting in a mimicking of the soft-tissue interface of natural teeth with a true papilla. This requires a connection between implant and fiber at high angles, which may be achieved via supracrestal fibers.

As discussed above, the dental implant of the exemplary embodiment may be used for treating “black triangles” which may be particularly problematic between two adjacent implants. Thus, references to an “adjacent tooth” in the description of the exemplary embodiments may refer to an adjacent implant. It will be understood by those skilled in the art, however, that references to an adjacent tooth should not be limited to dental implants. It will also be understood by those skilled in the art that reference to the term “supracrestal fibers,” as used herein, is intended to refer to sub-micron diameter collagen fibrils containing only collagen molecules, water, and ions.

As shown in FIG. 1, a dental implant 100 for facilitating growth or interdental papilla comprises an implant portion 102 configured to be inserted into a bone of a jaw of the patient, an abutment 104 at an exposed end 106 of the implant portion 102, and a crown 108 attached to the abutment 104. In one exemplary embodiment, the implant portion 102 may be configured as a screw threadable into the jaw of the bone in a position along the bone in which a tooth is to be replaced. In an exemplary embodiment, upon threading of the implant portion 102 into the bone, a user (e.g., dental surgeon) may affix the abutment 104 to the exposed end 106 of the implant portion 102.

Once the abutment 104 is attached to the implant portion 102, a crown 108 (i.e., artificial tooth) is then placed over the abutment 104 and adhered thereto. It will be understood by those skilled in the art that in some exemplary embodiments, the dental implant 100 may have an integrated screw as the implant portion 102 and the abutment 104. In this embodiment, the abutment 104 (and/or the implant portion 102) may be formed of a material such as, for example, titanium or any other material suitable for construction of a dental implant abutment, and includes supracrestal fibers 110 extending substantially perpendicularly from an exterior surface 112 of the abutment 104 so that, upon implantation of the dental implant 100 into the bone, the supracrestal fibers 110 form a scaffold for building up the papilla between the dental implant 100 and an adjacent tooth. The supracrestal fibers 110 form a scaffold to treat “black triangles” (see FIG. 2) that may form between adjacent teeth when the gums do not sufficiently fill the space between the teeth—e.g., when the interdental papilla surrounding the dental implant 100 is compromised. In one particular embodiment, the supracrestal fibers 110 may be utilized for building up papilla between two adjacent implants.

The supracrestal fibers 110, which extends perpendicularly from the exterior surface of the abutment 104, will tent up the soft tissue adjacent to the dental implant 100 in a supracrestal region of the dental implant 100 thus providing a solution to compromised papilla that forms in the absence of the fibers. This configuration is superior to the dental implants which are only supported via the alveolar bone because it more closely mimics a natural tooth which does have fibers superior to the alveolar bone which supports the tooth. The papilla formed in the presence of the supracrestal fibers 110 is more cosmetically similar to a gum line of natural teeth. In addition, it may provide a superior barrier against bacterial invasion that frequently results in chronic periodontitis with current implants.

As published in, “A Bottom-Up Approach Grafts Collagen Fibrils Perpendicularly to Titanium Surfaces,” ACS Appl. Bio Mater. 2020 3 (9), 6088-6095, it has recently been discovered that collagen molecules may be covalently attached end-on to titanium surfaces. Collagen is a long molecule (˜290 nm) consisting of three individual strands of amino acids braided together in a helix. We can think of this helix as being modeled as a cylinder. There are two relevant surfaces in this picture: one is the circular end of the cylinder and the other is the long surface that connects the two ends.

Previously, collagen was covalently attached to other surfaces and other molecules with reactions that utilize carboxylic acids (COOH) and amine (NH2) groups on amino acid residues. The COOH groups are found on glutamic and aspartic acids as well as at the carboxy terminus of the amino acid strands. The NH2 groups are found on lysine and arginine as well as the amino terminus of the amino acid strands. Each molecule of type I collagen contains 287 COOH groups and 180 NH2 groups. Almost all of these are found on the long surface connecting the two ends. Only three COOH and three NH2 groups are located at the ends of the molecule. Thus, these chemistries, just by virtue of the number of possible locations, attach collagen molecules along the stretch of surface connecting the two ends. The resulting geometries look similar to a pile of cooked spaghetti noodles laying on a plate. The molecules (noodles) all lie roughly parallel to the surface of the plate.

FIG. 3, however, shows an overview of the method used to create collagen fibrils perpendicular to a surface of an implant such as, for example, a surface formed of titanium. The method involves converting some of the terminal carboxylate groups into ketones (C═O). These are unique functional groups on a collagen molecule. They don't exist naturally on amino acids. This makes it possible to do chemistry only with these terminal ketones and not on any of the carboxylates or amines along the long stretch of the molecule.

This method was modified for use with collagen molecules and titanium surfaces. In particular, upon attachment of individual molecules, they were used as nucleation sites to grow collagen fibrils. Because the original attached molecules were oriented end-on to the surface, the resulting fibrils of collagen self-assembled in a direction perpendicular to the surface. This process can be described in two steps. First, a nucleus must form. This is the smallest number of collagen molecules associated with each other that is stable. Collections of molecules smaller than this are just as likely to separate from each other as they are to grow into a larger fibril.

The second step is growth. After nucleation occurs, either more molecules add individually onto the nucleus or multiple nuclei aggregate together to grow a larger fibril. The nucleation step is separated from the growth step during synthesis. If these steps are not separated (which is the standard method used to self-assemble collagen fibrils from molecules), then large gelatinous assemblies of randomly oriented fibrils would have resulted. This would happen because, in the solution above the surface containing the end-on collagen molecules, many molecules exist having random orientations. Each of these is a potential nucleation site. To avoid the random end product, nucleation is conducted for a short time and the volume is subsequently rinsed out so that all of the randomly oriented fibrils above the surface are removed from the system in this rinsing step.

The growth step requires much lower concentrations of collagen molecules in solution compared to the nucleation step. So, after nuclei have formed on the surface and solution phase nuclei are removed, the surface is exposed to a solution containing too few molecules to allow nucleation to occur but enough molecules to allow growth to occur. This results in fibrils of self-assembled collagen oriented roughly perpendicularly to the titanium surface. The conventional methods for attaching molecules and fibrils to surfaces result in fibrils that make only very small angles with a surface. This new method, however, allows for fibrils that may take on a much wider range of angles anywhere from near parallel to the surface to perfectly perpendicular to the surface.

One or more of the supracrestal fibers 110 of the exemplary embodiment may be similarly formed on the exterior surface 112 of the abutment 104 of the dental implant 100, at a high angle—e.g., substantially perpendicular to the exterior surface 112. According to an exemplary embodiment, the supracrestal fibers 110 may be formed on the exterior surface 112 at an angle ranging between 60 degrees and 90 degrees and, in a preferred embodiment, may range from between 80 degrees and 90 degrees. In an exemplary embodiment, the exterior surface 112 of the abutment 104 may be formed of a titanium material. It will be understood by those skilled in the art, however, that the exterior surface 112 is not limited to a titanium surface and that the abutment 104 may be formed of any of a variety of materials suitable for use in a dental implant.

The exterior surface 112 of the abutment 104 in the exemplary embodiment, however, is highly polished and machined, and is configured to interface with the soft tissue rather than be implanted (i.e., inserted) into the bone. It will be understood by those skilled in the art that attachment to a smooth, machined surface such as the exterior surface 112 of the abutment 104 is different than attachment to a roughened surface. While the part of the dental implant 100 that osseointegrates with the bone (e.g., the implant portion 102) is roughened, the surface of the supracrestal area is polished to prevent bone from creeping up to the level of the abutment 104. According to the exemplary embodiment, a scaffold is created above the bone, but below the intraoral space. When you don't have bone growing up, you also lose to some extent, the other components that come with bone—the vascularity, the bone morphogenetic proteins, everything that comes with bone and bone marrow. According to the exemplary embodiment, a scaffold is created for the gum area above the bone, but below the intraoral space.

Since the supracrestal fibers 110 of the present disclosure are attached to the abutment 104 rather than the implant portion 102 (e.g., a screw), the supracrestal fibers 110 do not have to withstand all of the force that goes through the tooth and into the bone at the root of the tooth. The supracrestal fibers 110 are thus required to withstand load only sufficient to tent up soft tissue around the tooth. This allows clinical success with a much lower level of covalent coupling of the fibers attached to the titanium compared with applications in the bony attachment portion of the device. With the application of at least one of the supracrestal fibers 110, having at least 1 covalent attachment, a reasonable chance of success exists because the mechanical environment that it needs to sustain is very different. This may lead to different quality control (as compared to implants in which collagen fibers are attached to the portion of implant inserted into bone) with respect to ensuring that the fibers get attached properly and may lead to more rapid processing since it does not require as much time for all of the covalent attachments to occur.

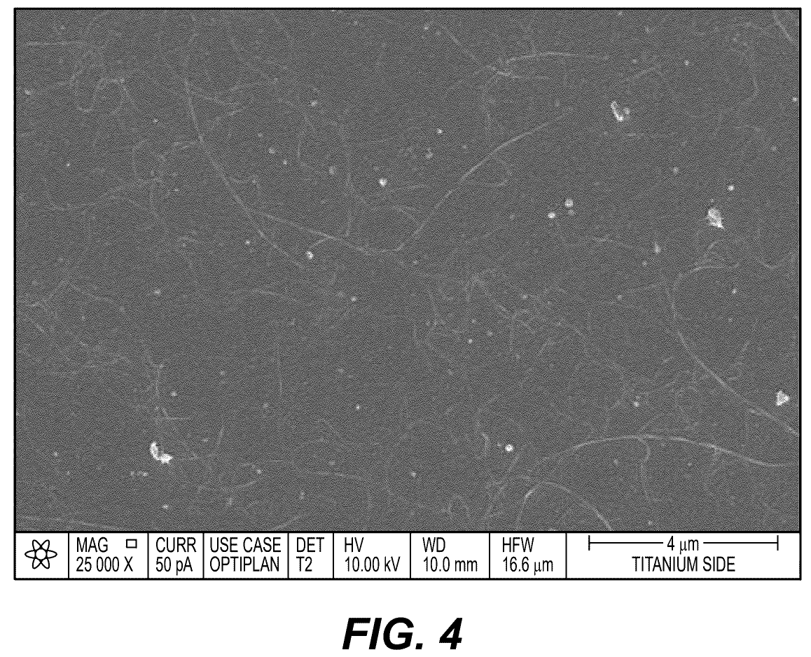

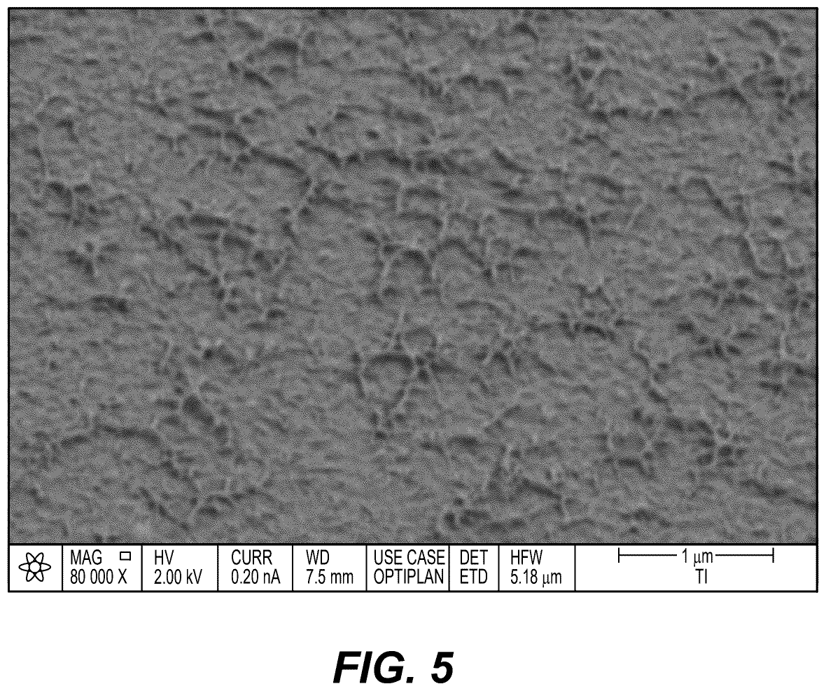

While a single covalent attachment to one of the supracrestal fibers 110 may, in some cases, be sufficient, it will be understood by those skilled in the art that, using the ketone-collagen technology described herein, the exterior surface 112 may be uniformly covered with fibrils 10, as shown in FIG. 4. While the supracrestal fibers 110 (e.g., lengths of a few microns to a few dozen microns) appear to be flat, it has been shown that these supracrestal fibers 110 have merely fallen during the dehydration step needed prior to scanning electron microscope (SEM) imaging and that the supracrestal fibers 110 are likely all standing while in solution. In particular, when growth conditions are reduced, many nubs are extending from the surface, as shown in FIG. 5. This is consistent with the idea that the exterior surface 112 with the supracrestal fibers 110 (e.g., long supracrestal fibers) actually have fibrils extending away from the exterior surface 112 when they are hydrated.

According to an exemplary embodiment, the dental implants 100 may be manufactured to include the supracrestal fibers 110 attached thereto. In an exemplary embodiment, the supracrestal fibers 110 will project substantially perpendicularly from all over the exterior surface 112, in all directions. As discussed above, however, the abutment 104 may include any number of the supracrestal fibers 110 and has a reasonable chance of success of forming a sufficient scaffold for facilitating papilla formation between adjacent teeth even with a single one of the supracrestal fibers 110.

It will be understood by those skilled in the art that since the abutment 104 may be manufactured to include the supracrestal fibers 110 thereon, the user (e.g., dental surgeon) may implant the dental implant 100 in a conventional manner determined by the user depending on a number of factors including, for example, the number of dental implants being inserted and a condition of the jawbone. In general, dental implant surgery is a multi-stage process performed over time. In some cases, dental implant surgery is performed in stages over several months.

The process for dental implantation may include damaged tooth removal, preparation of the bone, placement of the implant portion 102 (e.g., screw), placement of the abutment 104, and finally placement of the crown 108 (i.e., artificial tooth). Upon placement of the abutment 104, the supracrestal fibers 110 form a scaffold between the dental implant 100 and the adjacent tooth—along the soft tissue between the dental implant and the adjacent tooth—to facilitate growth of the interdental papilla. This process may take anywhere between 12 weeks to 9 months depending on the severity of the pre-existing defect. Once the interdental papilla is formed, as desired, the crown 108 may be placed.

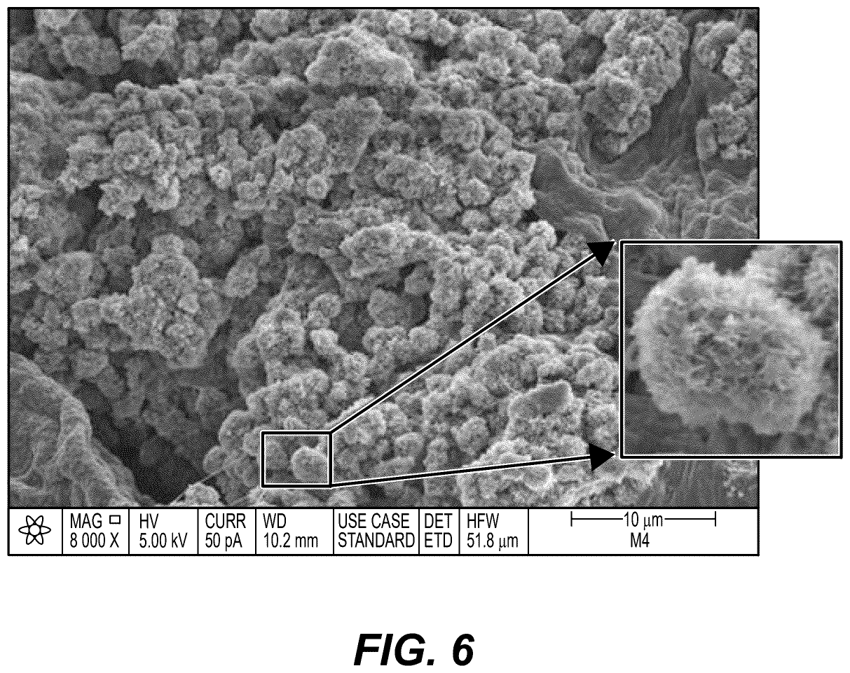

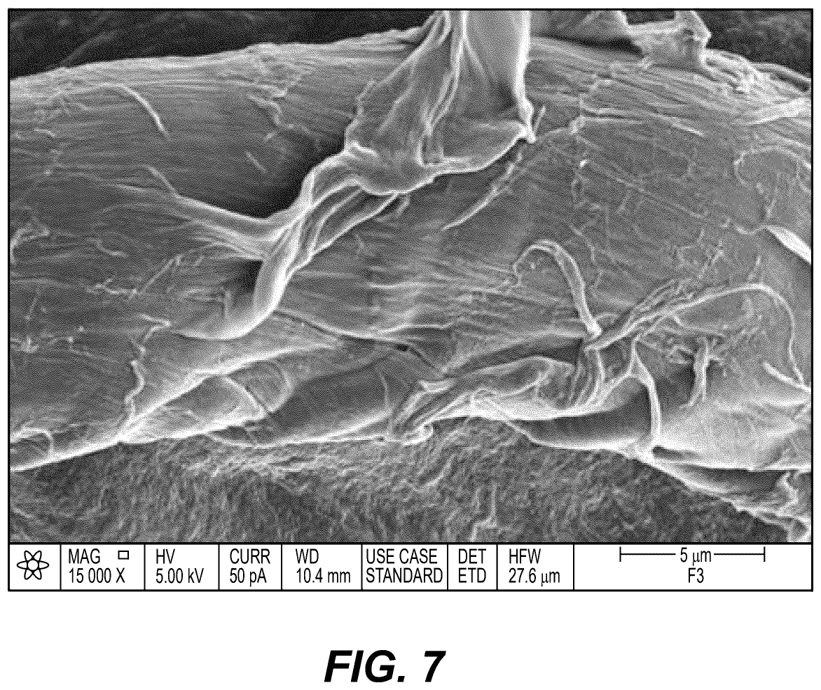

In another embodiment, the supracrestal fibers 110 may be mineralized using, for example, fetuin. Fetuin allows the supracrestal fibers 110 to be mineralized without creating substantial extrafibrillar mineral deposits. As will be understood by those skilled in the art, bone mineral is substantially intrafibrillar, so that this is a more biomimetic results than other mineralized collagen products currently on the market. For example, FIG. 6 shows mineralized collagen fibrils without fetuin, which include extrafibrillar mineral deposits while FIG. 7 shows mineralized collagen with fetuin added. Both samples should show an increase in weight over unmineralized controls and both showed Ca and P signals by energy dispersal x-ray spectroscopy (EDS). The mineralized collagen with fetuin may integrate better with bone to create mineralized gels that may help regrow bone in, for example, challenging situations such as where lateral bone growth is needed or in diabetic patients where bone growth can be suppressed. It will also be understood by those skilled in the art that supracrestal fibers that reach from the abutment 104 down into bone may osseointegrate better using this technology.

According to a further embodiment, the supracrestal fibers 110 may also be utilized as drug delivery vehicles. The ketone-collagen linker technology described herein may also facilitate attachment of collagen gels to surfaces, providing a platform for collagen gel-based drug delivery methods to deliver growth factors for healing and/or antibiotics with long term slow release to aid in peri-implantitis and/or analgesia. For example, antibiotics or growth factors may be incorporated with the supracrestal fibers to facilitate integration thereof into the soft tissue. Collagen gels attached to surfaces using the methods described herein have been shown to remain attached to the surface even upon being exposed to shear while less than 5 percent of collagen gels without the ketone collagen attachment have been shown to remain after shearing. In a further embodiment, the supracrestal fibers 110 may include a long term and/or reloadable release mechanism so that the drug may be delivered over an extended period of time.

It will be apparent to those skilled in the art that various modifications may be made in the present disclosure, without departing from the scope of the disclosure. Furthermore, those skilled in the art will understand that the features of any of the various embodiments may be combined in any manner that is consistent with the description and/or the functionality of the embodiments.

What is claimed is:

1. A dental implant for facilitating interdental papilla growth, comprising:
an implanted portion configured to be inserted into a jawbone;
an abutment configured to be attached to an exposed end of the implanted portion; and
supracrestal fibers extending laterally from an exterior surface of the abutment to form a scaffold between the dental implant and an adjacent tooth for facilitating interdental papilla growth in a supracrestal region of the dental implant.

2. The dental implant of claim 1, wherein the supracrestal fibers are covalently attached to the abutment to extend substantially perpendicularly from the exterior surface of the abutment.

3. The dental implant of claim 1, wherein the implanted portion is configured as a screw threadable into the jawbone.

4. The dental implant of claim 1, further comprising a tooth-shaped crown configured to be mounted over the abutment and adhered thereto.

5. The dental implant of claim 1, wherein the abutment is formed of titanium.

6. The dental implant of claim 1, wherein the abutment includes a smooth, machined surface.

7. The dental implant of claim 1, wherein the implanted portion and the abutment are integrally formed.

8. The dental implant of claim 1, wherein the supracrestal fibers are mineralized using fetuin.

9. The dental implant of claim 1, wherein the supracrestal fibers are incorporated with one of growth factors, analgesics and antibiotics.

10. A method for dental implantation, comprising inserting an implanted portion into a jawbone, an abutment attached to an exposed end of the implanted portion so that the abutment extends exterior to the jawbone and supracrestal fibers extend laterally from the abutment to form a scaffold between the abutment and an adjacent tooth to facilitate growth of interdental papilla in a supracrestal region.

11. The method of claim 10, wherein the supracrestal fibers are covalently attached to the abutment to extend substantially perpendicularly from an exterior surface of the abutment.

12. The method of claim 10, wherein inserting the implanted portion includes threading a screw of the implanted portion into the jawbone.

13. The method of claim 10, further comprising mounting a tooth-shaped crown over the abutment and adhering the tooth-shaped crown thereto.

14. The method of claim 10, wherein the abutment is formed of titanium.

15. The method of claim 10, wherein the abutment includes a smooth, machined surface.

16. The method of claim 10, wherein the abutment is attached to the implanted portion after implantation of the implanted portion into the jawbone.

17. The method of claim 10, wherein the implanted portion and the abutment are integrally formed.

18. The method of claim 10, wherein the supracrestal fibers are mineralized using fetuin.

19. The method of claim 10, wherein the supracrestal fibers are incorporated with one of growth factors, analgesics and antibiotics.
